(19) United States
(12) Patent Application Publication
(10) Pub. No.: US 2017/0189187 A1
(43) Pub. Date: Jul. 6, 2017

(54) METHOD OF REPAIRING A HEART VALVE

(71) Applicant: Hangzhou DiNovA Medtech Co., Ltd., Hangzhou (CN)

(72) Inventors: Carlos E. Ruiz, New York, NY (US); Tingchao Zhang, Shenzhen (CN); Yongsheng Wang, Shenzhen (CN); Zhenjun Zi, Shanghai (CN)

(21) Appl. No.: 14/985,469

(22) Filed: Dec. 31, 2015

(51) Int. Cl. A61F 2/24; A61B 17/04

(52) U.S. Cl. CPC A61F 2/2454; A61F 2/2466; A61B 17/0401; A61B 17/0487

(57) ABSTRACT

A method of repairing the heart valve is described. The method includes securing a first tissue anchor to a first posterior portion of annulus between posterior and anterior commissures; securing a second tissue anchor to a first anterior portion of the annulus between the posterior and anterior commissures; and securing a third tissue anchor to a second anterior portion of annulus between the posterior and anterior commissures. At least one tensile member is spanned between the first, second and third tissue anchors and across the orifice of the heart valve. When tension is applied to the at least one tensile member, the posterior portion of the annulus is pulled toward the anterior portion of the annulus.

### TECHNICAL FIELD

The present application generally relates to a surgical method of heart valve, more particularly, relates to a method of repairing a heart valve.

### BACKGROUND

The mitral heart valve is composed of valve leaflets, or flaps of tissue, that open and close tightly to ensure that the flow of blood through the heart is in one direction only. The leaflets are held in position by a ring of tissue, the annulus, surrounding and attaching the leaflets to the walls of the heart between the left atrium and left ventricle. Chordae tendineae are tendons that tether the leaflets to papillary muscles within the left ventricle, which prevent the leaflets from prolapsing into the left atrium. A dysfunction of any one of these portions of the mitral heart valve anatomy can cause mitral regurgitation, or the partial backflow of blood from the left ventricle into the left atrium. Depending on the severity of the condition, the individual may experience a range of symptoms, including shortness of breath, pulmonary edema, or decreased exercise tolerance.

Surgical procedures may be used for reducing mitral regurgitation. Some of these procedures have included plicating the mitral heart valve tissue in order to reduce the size of the orifice created between the leaflets. One such surgical procedure, annuloplasty, is particularly useful in treating mitral heart valve regurgitation. Annuloplasty modifies the annulus, through one or more plications, and this can return the valve to a functional geometry.

However, many annuloplasty procedures are highly invasive and may incorporate open heart surgery, which poses significant risk to the patient. Therefore, there is a need for a less invasive approach for plicating tissue by eliminating the need for open heart surgery while returning the mitral heart valve to a functional geometry.

### SUMMARY OF THE INVENTION

In one illustrative embodiment of the present invention, a method of repairing the heart valve is described. The method includes securing a first tissue anchor to a first posterior portion of annulus between posterior and anterior commissures; securing a second tissue anchor to a first anterior portion of the annulus between the posterior and anterior commissures; and securing a third tissue anchor to a second anterior portion of annulus between the posterior and anterior commissures. At least one tensile member is spanned between the first, second and third tissue anchors and across the orifice of the heart valve. When tension is applied to the at least one tensile member, the posterior portion of the annulus is pulled toward the anterior portion of the annulus.

In another illustrative embodiment of the present invention, a second method of repairing the mitral heart valve is described. This second method includes directing a guide-wire into the left ventricle, across a first posterior portion of the annulus, through the left atrium, across a second anterior portion of the annulus, and then across a second anterior portion of the annulus, and then returning into the left ventricle. A first tissue anchor is directed along the guide-wire to the first posterior portion of the annulus and secured. A second tissue anchor is then directed along the guide-wire to the first anterior portion of the annulus and secured. A third tissue anchor is then directed along the guide-wire to the second anterior portion of the annulus and secured. At least one tensile member is spanned between the first, second and third tissue anchors and across the orifice of the mitral heart valve. When tension is applied to the at least one tensile member, the posterior portion of the annulus is pulled toward the anterior portion of the annulus.

In a further embodiment, the at least one tensile member is a suture.

In a further embodiment, the tension applied to the at least one tensile member is maintained with a suture locker.

In a further embodiment, the suture locker is percutaneously directed to the at least one tensile member with at least one catheter.

In a further embodiment, each of the first, second and third tissue anchors includes a plurality of discrete, flat, flexible anchor elements coupled by the at least one tensile member, respectively.

In a further embodiment, each of the first, second and third tissue anchors is secured by advancing at least two of the plurality of discrete, flat, flexible anchor elements through to one side of the annulus with at least one of the discrete, flat, flexible anchor elements disposed on the other side of the annulus.

In a further embodiment, the first tissue anchor is percutaneously directed to the first posterior portion of annulus with at least one catheter, the second tissue anchor is percutaneously directed to the first anterior portion of the annulus with the at least one catheter, the third tissue anchor is percutaneously directed to the second anterior portion of the annulus with the at least one catheter.

In one preferable illustrative embodiment of the present invention, the heart valve is a mitral heart valve having posterior and anterior leaflets forming the orifice therebetween, the annulus surrounds the posterior and anterior leaflets which join the annulus at the posterior and anterior commissures, wherein the posterior leaflet includes P1, P2, and P3 regions and the anterior leaflet includes A1, A2, and A3 regions, the first anterior portion of the annulus being near the A1 region, the second anterior portion of the annulus being near the A3 region, and the first posterior portion of the annulus being near the P2 region.

In one preferable illustrative embodiment of the present invention, the heart valve is a tricuspid heart valve having a posterior leaflet, an anterior leaflet, and a septal leaflet. A first tissue anchor is arranged at the posterior leaflet, the second tissue anchor is arranged at a first region of anterior leaflet, the third tissue anchor is arranged at the second region of anterior leaflet.

### BRIEF DESCRIPTION OF THE DRAWINGS

So as to further explain the invention, an exemplary embodiment of the present invention will be described with reference to the below drawings, wherein:

FIG. 1 is a flow chart showing successive steps of one exemplary procedure for repairing a mitral heart valve according to present application.

FIG. 2 is a cross-sectional view illustrating successive steps of one exemplary procedure for advancing and securing the first tissue anchors.

FIG. 3 is a top view illustrating the mitral heart valve from the left atrium after tissue plication and with the first, second and third tissue anchors positioned at the A1, A3 and P2 regions.

FIG. 4 is a top view illustrating the mitral heart valve from the left atrium after tissue plication and with a first staple positioned between the A1, A3 and P2 regions.

FIG. 5 is a top view illustrating the tricuspid heart valve having a posterior leaflet, an anterior leaflet, and a septal leaflet.

FIG. 6 is a top view illustrating the tricuspid heart valve having a posterior leaflet, an anterior leaflet, and a septal leaflet.

### DETAILED DESCRIPTION OF THE PREFERRED EMBODIMENT

These and other advantage, aspect and novel features of the present invention, as well as details of an illustrated embodiment thereof will be more fully understood from the following description and drawings, while various embodiments of the present invention are presented by way of examples only, not limitation.

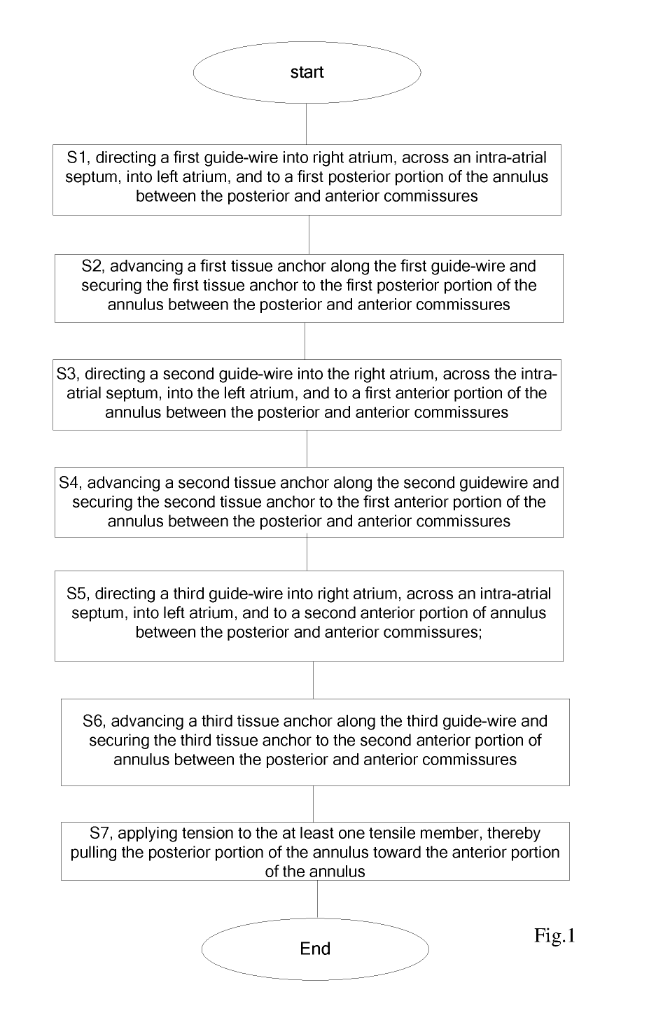

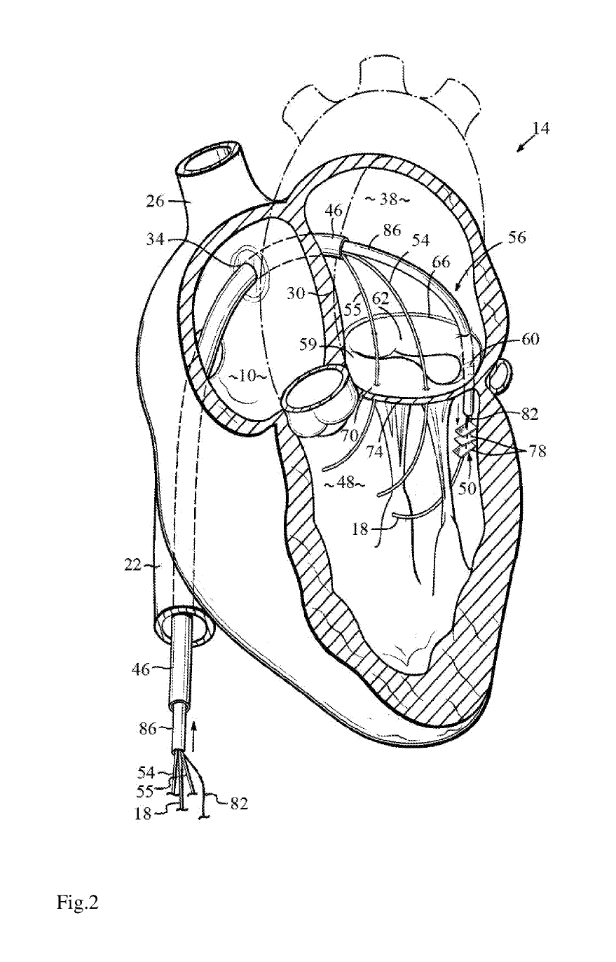

FIG. 1 is a flow chart showing successive steps of one exemplary procedure for repairing a mitral heart valve according to present application. FIG. 2 is a cross-sectional view illustrating successive steps of one exemplary procedure for advancing and securing the first tissue anchors. FIG. 3 is a top view illustrating the mitral heart valve from the left atrium after tissue plication and with the first, second and third tissue anchors positioned at the A1, A3 and P2 regions.

In step S1, as shown in FIG. 2, the method begins by percutaneously accessing the right atrium 10 of the heart 14 from a suitable venous access site. The venous access site can be located near the jugular vein, superiorly, from the femoral vein, inferiorly, or from other suitable superficial veins.

In step 2, a first guide-wire 18 is directed into the venous access site, through the inferior or superior vena cava 22, 26, as appropriate, and into the right atrium 10. Suitable guide-wires 18 can include commercially-available guide-wires commonly used in catheter-based procedures, including steerable guide-wires. The first guide-wire 18 can then be directed across the intra-atrial septum 30, for example near the fossa ovalis 34, and into the left atrium 38 in accordance with known transseptal procedures.

The first guide-wire 18 can be directed to a first posterior portion of the annulus (i.e. the posterior annulus 66) between the posterior and anterior commissures 59, 60. One skilled in the art may generally refer to the illustrated position as the P2 region, which is located medially at the base of the posterior leaflet 62 and proximal to the intra-atrial septum 30. Another suitable position could be the P3 region, which is located laterally at the base of the posterior leaflet 62 and proximal to the intra-atrial septum 30. Another suitable position could be the P1 region, which is located laterally at the base of the posterior leaflet 62 and proximal to the anterior commissure 60. Another suitable position could be any region near the P1, P2 or P3 region.

Once the first guide-wire 18 is directed to the desired position on the posterior annulus 66, the first guide-wire 18 is then advanced across the posterior annulus 66 and into the left ventricle 48.

After the first guide-wire 18 is in position, a guide catheter 46 can be advanced over the first guide-wire 18 and into the left atrium 38. The guide catheter 46 can be any suitable catheter that can be directed through the vascular system to aid in the delivery of subsequent surgical devices, such as tissue anchors 50 for use with the procedures described herein. Though not specifically shown, a physician can also use additional surgical instruments, such as an obturator, to sufficiently dilate the puncture through the intra-atrial septum 30 to accommodate the larger diameter guide catheter 46.

In step S2, the first tissue anchor 50 is directed along the first guide-wire 18, across the posterior annulus 66, and into the left ventricle 48 and then secured to or near the P2 region of the posterior annulus 66.

Turning now to FIG. 2, where the advancement and deployment of the first tissue anchor 50 is shown and described. While any tissue anchor device known in the art can be used, including but not limited to clips, wires, or staples, the particular tissue anchor device shown is collapsible and comprises a plurality of discrete, flat, flexible anchor elements 78 coupled by a flexible tensile member 82. The anchor elements 78 can be formed from a surgical grade fabric material (e.g., a polyester material such as DACRON) designed to promote tissue in-growth so that the anchor 50 becomes essentially encased in tissue over time. The anchor elements 78 are coupled to the tensile member 82, in this example a suture, by threading the suture upwardly through the anchor elements 78 and then back downwardly through the anchor elements 78. A slip knot is then formed, or another type of lock member is used, so that when a proximal end portion of the tensile member 82 is pulled, all of the anchor elements 78 will be drawn together against opposite sides of the annular tissue.

In use, the first tissue anchor 50 with a delivery sheath 86 is directed along the first guide-wire 18, across the posterior annulus 66, and into the left ventricle 48. The first tissue anchor 50 is then at least partially deployed from the delivery sheath 86 on the left ventricular side of the posterior annulus 66. As necessary, the first guide-wire 18 can be removed before or after the tissue anchor deployment process. Once a sufficient portion of the first tissue anchor 50 has been deployed within the left ventricle 48, the physician can stop deploying the anchor elements 78, slightly retract the delivery sheath 86 back across the posterior annulus 66 into the left atrium 38, and then deploy the remainder of the anchor elements 78 of the tissue anchor 50 within the left atrium 38.

In step 3, with the guide catheter 46 positioned within the left atrium 38, a second guide-wire 54 can then be directed through the lumen of the guide catheter 46 and into the left atrium 38.

In a similar manner, the second guide-wire 54 can be directed to a first anterior portion of the annulus (i.e. the anterior annulus 74) between the posterior and anterior commissures 59, 60. Once the second guide-wire 54 is directed to the desired position on the anterior annulus 74, the second guide-wire 54 is then advanced across the anterior annulus 74 and into the left ventricle 48.

In a similar manner, in step S4, the second tissue anchor 501 (FIG. 3) which is similar with the first tissue anchor 50 is directed along the second guide-wire 54, across the anterior annulus 74, and into the left ventricle 48 and then secured to or near the A1 region of the anterior annulus 74, which is located laterally at the base of the anterior leaflet 70 along the anterior annulus 74.

As described above, the second tissue anchor 501 is then at least partially deployed from the delivery sheath within the left ventricle 48, the delivery sheath is then retracted back across the anterior annulus 74, and the remainder of the second tissue anchor 501 is deployed within the left atrium 38.

In a similar manner, in step S5, the third guide-wire 55 can be directed to A3 region of the anterior annulus 74, which is located medially at the base of the anterior leaflet 70 and proximal to the intra-atrial septum 30. Once the third guide-wire 55 is directed to the desired position on the anterior annulus 74, the third guide-wire 55 is then advanced across the anterior annulus 74 and into the left ventricle 48.

In a similar manner, in step S6, the third tissue anchor 502 which is similar with the first tissue anchor 50 is directed along the third guide-wire 55, across the anterior annulus 74, and into the left ventricle 48, and then secured to or near the A3 region of the anterior annulus 74.

However, the procedure should not be considered limited to these regions of the posterior annulus 66 and anterior annulus 74, as one or more regions may be chosen depending on the location of the enlarged orifice through the orifice of the mitral heart valve 56. For example, if the posterior and anterior leaflets 62, 70 do not coapt at a lateral region of the mitral heart valve 56, then the repair can be directed to the A1, A3 to P1 regions; and if the posterior and anterior leaflets 62, 70 do not coapt medially, then the repair can be directed more appropriately to the A1, A3 to P3 regions.

Then in step S7, with all the first, second and third tissue anchors 50, 501 and 502 secured to their respective positions on the posterior and anterior portions of the annulus 66, 74, respectively, the physician can then plicate the tissue by pulling on the respective proximal end portions of the tensile members 82, 94 such that the posterior annulus 66 is pulled toward the anterior annulus 74.

The plication and position of the tissue can be maintained by directing a suture locker 98 along the tensile members 82, 94 to the surgical site. The advancing of the suture locker 98 can be accomplished with a delivery catheter in accordance with known methods.

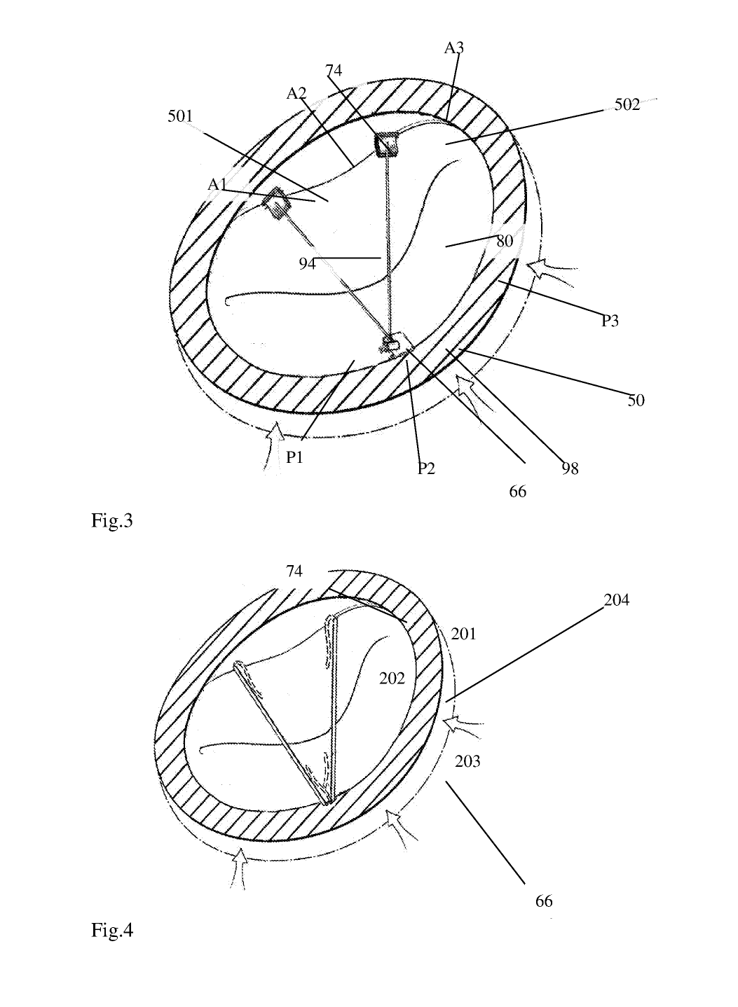

Alternatively, FIG. 4 illustrates the use of first and second legs 201, 202, 203 and the base of the staples as the tissue anchor and tensile members, respectively. The first, second and third staples can be positioned in a manner that is similar to the tissue anchors in FIG. 3 to pull the posterior annulus 66 toward the anterior annulus 74 and effectuate mitral heart valve repair.

According to present application, three tissue anchors are arranged at the posterior annulus 66 and the anterior annulus 74, in which two tissue anchors are arranged at the same annulus, and the third one is arranged at the different annulus. According to present application, the position of the third tissue anchor can be accurately adjusted according to the regurgitation position, so that excessive suture can be avoided, little regurgitation after the repair procedure would be resulted and the two leaflets are closer to each other.

According to our study, we find that, when arranging four tissue anchors with two at the posterior annulus 66 and other two at the anterior annulus 74, there may be a longer distance between the two tissue anchors at the posterior annulus 66, which may results in tissue protruding, thus forming leak at the middle in the posterior annulus. However, by arranging three tissue anchors, these defects would be overcome. As one suture is saved, less devices and less tensile force are required. Then, the required pull force is decreased and will not be exploded into the derelict parallel with the commissures. In additional, when employing the method of repairing the mitral heart valve according to present application, the distortion of the related tissue would be less, and no suture would be carried out on the health position, such that the function of which is maintained.

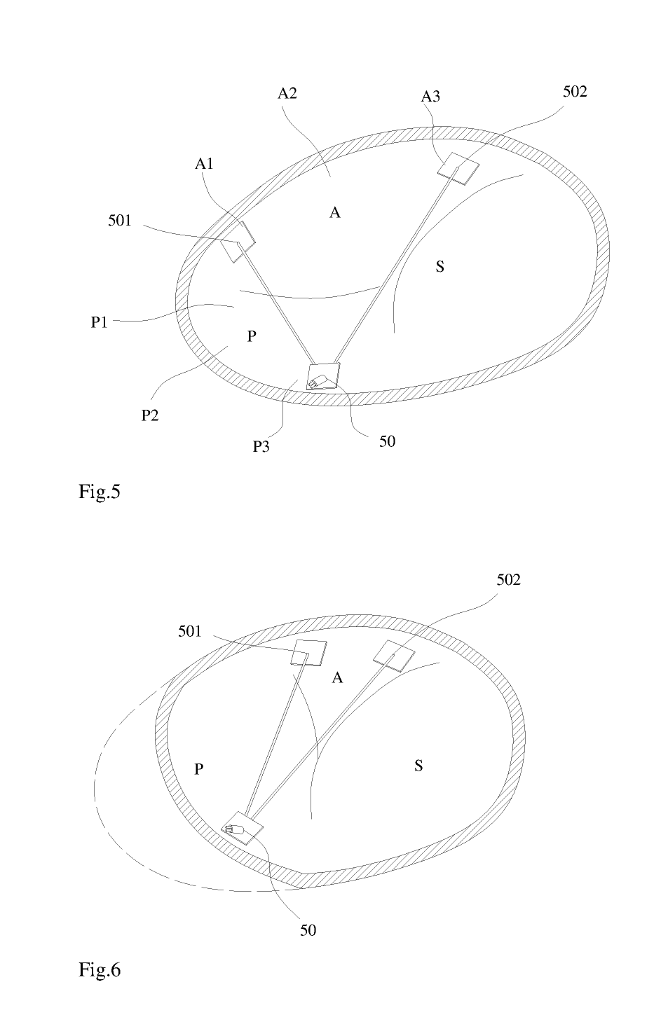

One skilled in the art should understand that, the method of repairing a mitral heart valve discussed above also can be used to repair a tricuspid heart valve. The detail of the processing steps of repairing the tricuspid heart valve can be obtained by one skilled in the art based on the disclosure of present application, and not described for concision. FIG. 5 is a top view illustrating the tricuspid heart valve with a posterior leaflet, an anterior leaflet, and a septal leaflet and FIG. 6 shows that the first, second and third tissue anchors are pulling together.

As shown in FIG. 5-6, the tricuspid heart valve comprises a posterior leaflet, an anterior leaflet, and a septal leaflet. In present application, the first tissue anchor 50 is arranged at the posterior leaflet, the second tissue anchor 501 is arranged at a first region of anterior leaflet, the third tissue anchor 502 is arranged at the second region of anterior leaflet. When repairing the tricuspid heart valve, all of the tissue anchors will be drawn together against opposite sides of the annular tissue, as shown in FIG. 6.

While the present invention has been described with reference to several embodiments, it will be understood by those skilled in the art that various changes may be made and equivalents may be substituted without departing from the scope of the present invention. In addition, many modifications may be made to adapt a particular situation or material to the teachings of the present invention without departing from its scope. Therefore, it is intended that the present invention not be limited to the particular embodiment disclosed, but that the present invention will include all embodiments falling within the scope of the appended claims.

1. A method of repairing a heart valve, comprising:
(a) securing a first tissue anchor comprising at least one tensile member to a first posterior portion of annulus between posterior and anterior commissures;
(b) securing a second tissue anchor comprising at least one tensile member to a first anterior portion of the annulus between the posterior and anterior commissures;
(c) securing a third tissue anchor comprising at least one tensile member to a second anterior portion of annulus between the posterior and anterior commissures; and
(d) applying tension to the at least one tensile member, thereby pulling the posterior portion of the annulus toward the anterior portion of the annulus.

2. The method according to claim 1, wherein the at least one tensile member is a suture.

3. The method according to claim 1, further comprising: maintaining the tension applied to the at least one tensile member with a suture locker.

4. The method according to claim 3, further comprising: percutaneously directing the suture locker to the at least one tensile member with at least one catheter.

5. The method according to claim 1, wherein each of the first, second and third tissue anchors includes a plurality of discrete, flat, flexible anchor elements coupled by the at least one tensile member, respectively.

6. The method according to claim 5, wherein each of the first, second and third tissue anchors is secured by advancing at least two of the plurality of discrete, flat, flexible anchor elements through to one side of the annulus with at least one of the discrete, flat, flexible anchor elements disposed on the other side of the annulus.

7. The method according to claim 1, further comprising: percutaneously directing the first tissue anchor to the first posterior portion of annulus with at least one catheter; and percutaneously directing the second tissue anchor to the first anterior portion of the annulus with the at least one catheter, percutaneously directing the third tissue anchor to the second anterior portion of the annulus with the at least one catheter.

8. The method according to claim 1, wherein the heart valve is a mitral heart valve having posterior and anterior leaflets forming the orifice therebetween, the annulus surrounds the posterior and anterior leaflets which join the annulus at the posterior and anterior commissures, wherein the posterior leaflet includes P1, P2, and P3 regions and the anterior leaflet includes A1, A2, and A3 regions, the first anterior portion of the annulus being near the A1 region, the second anterior portion of the annulus being near the A3 region, and the first posterior portion of the annulus being near the P2 region.

9. The method according to claim 1, wherein the heart valve is a tricuspid heart valve having posterior, septal and anterior leaflets, the first tissue anchor is arranged at the posterior leaflet, the second tissue anchor is arranged at a first region of anterior leaflet, the third tissue anchor is arranged at the second region of anterior leaflet.

10. A method of repairing a mitral heart valve, the mitral heart valve comprising posterior and anterior leaflets forming an orifice therebetween, an annulus surrounding the posterior and anterior leaflets, and posterior and anterior commissures where the posterior and anterior leaflets join the annulus, the method comprising:
(a) directing a first guide-wire into right atrium, across an intra-atrial septum, into left atrium, and to a first posterior portion of the annulus between the posterior and anterior commissures;
(b) advancing a first tissue anchor along the first guide-wire and securing the first tissue anchor to the first posterior portion of the annulus between the posterior and anterior commissures;
(c) directing a second guide-wire into the right atrium, across the intra-atrial septum, into the left atrium, and to a first anterior portion of the annulus between the posterior and anterior commissures;
(d) advancing a second tissue anchor along the second guide wire and securing the second tissue anchor to the first anterior portion of the annulus between the posterior and anterior commissures;
(e) directing a third guide-wire into right atrium, across an intra-atrial septum, into left atrium, and to a second anterior portion of annulus between the posterior and anterior commissures;
(f) advancing a third tissue anchor along the third guide-wire and securing the third tissue anchor to the second anterior portion of annulus between the posterior and anterior commissures; and
(g) applying tension to the at least one tensile member, thereby pulling the posterior portion of the annulus toward the anterior portion of the annulus.

11. The method according to claim 10, wherein the at least one tensile member is a suture.

12. The method according to claim 10, further comprising: maintaining the tension applied to the at least one tensile member with a suture locker.

13. The method according to claim 12, further comprising: percutaneously directing the suture locker to the at least one tensile member with at least one catheter.

14. The method according to claim 10, wherein each of the first, second and third tissue anchors includes a plurality of discrete, flat, flexible anchor elements coupled by the at least one tensile member, respectively.

15. The method according to claim 10, wherein each of the first, second and third tissue anchors is secured by advancing at least two of the plurality of discrete, flat, flexible anchor elements through to one side of the annulus with at least one of the discrete, flat, flexible anchor elements disposed on the other side of the annulus.

16. The method according to claim 10, further comprising: percutaneously directing the first tissue anchor to the first posterior portion of annulus with at least one catheter; and percutaneously directing the second tissue anchor to the first anterior portion of the annulus with the at least one catheter, percutaneously directing the third tissue anchor to the second anterior portion of the annulus with the at least one catheter.

17. The method according to claim 10, wherein the posterior leaflet includes P1, P2, and P3 regions and the anterior leaflet includes A1, A2, and A3 regions, the first anterior portion of the annulus being near the A1 region, the second anterior portion of the annulus being near the A3 region, and the first posterior portion of the annulus being near the P2 region.
